(19) United States
(12) Patent Application Publication
(10) Pub. No.: US 2025/0044306 A1
(43) Pub. Date: Feb. 6, 2025

(54) Methods for Determining Opioid Responsiveness in the Treatment of Pain

(71) Applicant: East Carolina University, Greenville, NC (US)

(72) Inventors: Stefan Clemens, Greenville, NC (US); Kori Brewer, Greenville, NC (US)

(21) Appl. No.: 18/714,824

(22) PCT Filed: Nov. 30, 2022
(86) PCT No.: PCT/US2022/080666

§ 371 Date: May 30, 2024

Related U.S. Application Data
(60) Provisional application No. 63/284,246, filed on Nov. 30, 2021.

(51) Int. Cl. G01N 33/94

(52) U.S. Cl. CPC G01N 33/9486; G01N 2800/2842; G01N 2800/52; G01N 2800/7066

(57) ABSTRACT

The present inventive concept provides methods and tools for determining opioid responsiveness and overcoming opioid desensitization in pain including, but not limited to, acute pain, chronic pain, opioid resistant neuropathic pain, and spinal cord injury pain, by identifying the metabolomic profile of the dopaminergic system in opioid responsive and opioid non-responsive subjects.

### RELATED APPLICATION DATA

The present application claims priority from and the benefit of U.S. Provisional Patent Application No. 63/284,246, filed Nov. 30, 2021, the disclosure of which is hereby incorporated herein by reference in its entirety.

### RESERVATION OF COPYRIGHT

A portion of the disclosure of this patent document contains material, which is subject to copyright protection. The copyright owner, East Carolina University, Greenville, North Carolina, a constituent institution of the University of North Carolina, has no objection to the reproduction by anyone of the patent document or the patent disclosure, as it appears in U.S. Patent and Trademark Office patent files or records, but otherwise reserves all copyright rights whatsoever.

### FIELD

The present inventive concept is directed to methods for determining indicators of opioid responsiveness including, but not limited to, addiction, tolerance and effectiveness as well as methods and compositions of matter for addressing opioid resistance and efficacy in the treatment and management of pain.

### BACKGROUND

Despite intense investigation, successful treatment of pain associated with spinal cord injury (SCI) remains challenging. Treatment options for pain include antiepileptics, antidepressants and opioids, however, these all show limited efficacy (Warms, Turner et al. 2002, Masri and Keller 2012, Felix 2014, Gwak, Kim et al. 2016). Opioids, such as morphine, are commonly used because of their potent analgesic properties in treating severe acute pain but often are unable to fully manage SCI pain, have inherent safety concerns, and undesirable side effects (Warms, Turner et al. 2002, Masri and Keller 2012, Felix 2014, Gwak, Kim et al. 2016, Hatch, Cushing et al. 2018). In a clinical study using morphine to treat pain more than 50% of patients failed to respond to treatment and only 15% continued morphine treatment pain management for more than 12 months (Attal, Guirimand et al. 2002). However, even in patients who respond to morphine, long-term treatment is problematic because of the development of tolerance (Collin and Cesselin 1991, Taylor and Fleming 2001), and increased risk for dependence and addiction (Ballantyne 2007, O'Connor and Dworkin 2009). The morphine resistant state of SCI pain mimics morphine tolerance which involves dysfunction of overlapping μ-opioid receptor (MOR) and dopamine-receptor mediated cAMP/PKA second messenger pathways (Schmidt, Tambeli et al. 2002, Zhang, Zhang et al. 2008, Enoksson, Bertran-Gonzalez et al. 2012, Zhang, Zhang et al. 2012). MORs co-localize with both the inhibitory dopamine 3 receptor (D3R) and the excitatory dopamine 1 receptor (DIR) in dorsal horn neurons in the spinal cord, an area important for nociceptive processing (Rodgers, Yow et al. 2019). Thus, the relationship between μ-opioid receptors (MORs) and dopamine receptors may provide a potential synergistic therapeutic mechanism.

The current investigators have shown previously in a rodent model that only one third of SCI animals had an analgesic response to morphine (Rodgers, Lim et al. 2020). This is similar to clinical findings of morphine resistance with pain in humans (Attal, Guirimand et al. 2002). The current investigators have also shown previously that Dopamine D1 and D3 receptor modulators restore morphine analgesia and prevent opioid preference in a model of pain (Rodgers et al. 2019) and that in the animals that did not respond to morphine (morphine non-responders), the addition of dopamine receptor modulators (either a DIR antagonist or a D3R agonist) could restore the analgesic effect of morphine (Rodgers, Lim et al. 2020). Together with previous data, in which we showed that morphine resistance was associated with a loss of function of the dopamine D3 receptor and an upregulation of the D1 receptor (Brewer et al., 2014), the earlier findings demonstrate that the dopamine system plays an important role in post-SCI morphine resistance. However, these findings did not reveal the injury's direct effect on dopamine pathways post-SCI. Thus, dopamine's relationship with the opioid system provides an opportunity to restore morphine efficacy, but few studies have examined how SCI alters the dopamine system at the level of the spinal cord or supraspinally, and none have assessed the differences between SCI animals that respond to opioid analgesics versus those that do not.

The present inventive concept overcomes previous shortcomings in the art by providing methods and compositions of matter utilizing metabolic analysis to assess opioid responsiveness in the treatment and management of acute, intermittent and chronic pain.

### SUMMARY

In one aspect, the present inventive concept provides methods for determining indicators of opioid addiction, tolerance and effectiveness as well as methods and compositions for addressing opioid resistance in the treatment and management of acute, intermittent and chronic pain and further providing a personalized medicine approach to determine opioid dosing and/or duration of treatment.

Further aspects include methods of determining opioid responsiveness in a subject, the methods including determining a metabolomic profile present in a sample from the subject including analysis of specific agents and/or biochemical pathways, wherein the biochemical pathways are selected from the group consisting of biochemical pathways involving tyrosine, tryptophan, linoleic acid, phenylalanine, sphingolipid, glycerophospholipid, D-glutamine, D-glutamate, dopamine and L-DOPA, and combinations thereof.

Additional aspects include methods of overcoming opioid desensitization in a subject in need thereof, the methods including determining a metabolomic profile present in a sample from the subject including analysis of specific agents and/or biochemical pathways, wherein the biochemical pathways are selected from the group consisting of biochemical pathways involving tyrosine, tryptophan, linoleic acid, phenylalanine, sphingolipid, glycerophospholipid, D-glutamine, D-glutamate, dopamine and L-DOPA, and combinations thereof.

Aspects also include methods of providing enhanced analgesia in a subject, the methods including determining the metabolic profile present in a sample from the subject and administering to the subject an effective amount of an opioid when the subject is determined to be opioid responsive or suspected to be opioid responsive, or administering a non-opioid treatment when the subject is determined to be opioid non-responsive or suspected to be opioid non-responsive.

Further aspects include methods of classifying a subject as opioid responsive or opioid non-responsive, the methods including determining a metabolomic profile present in a sample from the subject including analysis of specific agents and/or biochemical pathways, wherein the biochemical pathways are selected from the group consisting of biochemical pathways involving tyrosine, tryptophan, linoleic acid, phenylalanine, sphingolipid, glycerophospholipid, D-glutamine, D-glutamate, dopamine and L-DOPA, and combinations thereof.

Additional aspects include kits including components to collect a sample from a subject and allow a transfer of the sample to a device or medium to allow analysis of the specific agents and/or biochemical pathways and/or agents thereof in the sample to obtain a metabolic profile of the subject's sample to predict opioid responsiveness as well as systems directed to collecting a sample from a subject and allowing a transfer of the sample to a device or medium to allow analysis of the specific agents and/or biochemical pathways and/or agents thereof in the sample to obtain a metabolic profile of the subject's sample to predict opioid responsiveness.

The foregoing and other aspects of the inventive concept are explained in greater detail in reference to the drawings and description set forth herein.

### BRIEF DESCRIPTION OF THE DRAWINGS

FIG. 1: Overview of animal numbers and testing paradigm.

FIG. 2A) SCI results in thermal hyperalgesia under control conditions and morphine (2 mg/kg) results in a dichotomous response. B) Percent analgesia results for SCI animals that are responsive to morphine under different drug conditions. C) Percent analgesia for SCI animals that are nonresponsive to morphine under different drug conditions. Mean±SEM for each group is shown.

FIG. 3: Spinal cord lesion size is not associated with morphine responsiveness. A) A representative longitudinal section of the epicenter of a contusion spinal cord injury. The dashed black lines denote how width and height were measured to calculate area. B) Estimated area for the epicenter of lesions for morphine responsive and nonresponsive SCI animals. C) Measured depth to lesion between morphine responsive and nonresponsive animals. Black triangles represent individual morphine responsive animals and gray circles represent morphine nonresponsive animals.

FIG. 4. A) Principal component analysis of metabolomics datasets of the striatum. Analysis was performed with MarkerView 1.3.1 and shows distinct clustering of groups. Open squares represent uninjured animals, triangles represent morphine responsive SCI animals and circles represent SCI animals that were nonresponsive to morphine. B) Graphical visualization of MetaboAnalyst comparing morphine responsive SCI animals versus morphine nonresponsive Sci animals. Each circle represents a different pathway with size and color indicating pathway impact and p-value (red being most significant). C) Graphical visualization of MetaboAnalyst comparing all SCI animals, regardless of morphine response, to uninjured animals.

FIG. 5. A) Principal component analysis of metabolomics datasets of the spinal cord. Principal component analysis was performed with MarkerView 1.3.1 and shows distinct clustering of groups. Open squares represent uninjured animals, triangles represent morphine responsive SCI animals and circles represent SCI animals that were nonresponsive to morphine. B) Graphical visualization of MetaboAnalyst comparing morphine responsive SCI animals versus morphine nonresponsive SCI animals. Each circle represents a different pathway with size and color indicating pathway impact and p-value (red being most significant). C) Graphical visualization of MetaboAnalyst comparing all SCI animals, regardless of morphine response, to uninjured animals.

FIG. 6: A) Dopamine levels in the striatum of morphine nonresponsive SCI animals are significantly reduced compared to morphine responsive SCI animals. B) Dopamine levels in the lumbar spinal cord of SCI animals (both morphine responsive and nonresponsive) are decreased compared to injured animals. Mean±SEM for each group is shown.

### DETAILED DESCRIPTION

The present invention will now be described with reference to the following embodiments. As is apparent by these descriptions, this invention can be embodied in different forms and should not be construed as limited to the embodiments set forth herein. Rather, these embodiments are provided so that this disclosure will be thorough and complete, and will fully convey the scope of the invention to those skilled in the art. For example, features illustrated with respect to one embodiment can be incorporated into other embodiments, and features illustrated with respect to a particular embodiment can be deleted from that embodiment. In addition, numerous variations and additions to the embodiments suggested herein will be apparent to those skilled in the art in light of the instant disclosure, which do not depart from the instant invention.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents, and other references mentioned herein are incorporated by reference in their entirety.

The current technology explores the changes in metabolomic profiles associated with pain, e.g., neuropathic pain, associated with spinal cord injury (SCI) as well as the effects of DIR and D3R modulators on analgesia in morphine-responsive and nonresponsive subjects after SCI with resultant respective metabolomic profiles. Through the differences now discovered regarding dopamine-related metabolomics and dopamine levels associated with opioid responsiveness after SCI, methods and diagnostic tools for addressing pain modulation and opioid tolerance are provided.

Embodiments of the present inventive concept include methods for determining indicators of opioid responsiveness such addiction, dependence, tolerance, and/or effectiveness as well as methods and compositions for addressing opioid resistance or desensitization in the treatment and management of acute, intermittent and chronic pain including providing a personalized medicine approach to determine opioid dosing and/or duration of treatment.

In some embodiments of the present inventive concept, an indicator is determined and used non-invasively and predicts prior to initiation of pain or analgesic treatment whether the subject will respond to an opioid. The indicator can be a single biomarker or a profile of biomarkers.

Embodiments of the present inventive concept also provide a Point-of-Care test that probes for indicators in a subject's sample (e.g., finger prick of blood, similar to a glucose test). A kit including components to collect the sample and/or testing strips or other media which can be read by a device to determine the subject's opioid-related metabolic profile are also provided. The kit can be used in both outpatient offices and hospital settings, where a patient has acute pain and opioid treatment is being considered. Notably, as a subject's response to opioids may change over time due to age, other medications, and/or lifestyle, the indicator test can be performed each time the patient seeks treatment.

According to further embodiments of the present inventive concept, a method of determining opioid responsiveness in a subject is provided. The method includes determining a metabolomic profile including analysis of specific agents, indicators or biomarkers, and biochemical pathways, wherein the biochemical pathways may be selected from the group consisting of biochemical pathways involving tyrosine, tryptophan, linoleic acid, phenylalanine, sphingolipid, glycerophospholipid, D-glutamine, D-glutamate, dopamine and L-DOPA, including, but not limited to, tyrosine synthesis pathway; tryptophan metabolism pathway; linoleic acid synthesis and metabolism; phenylalanine synthesis and metabolism; sphingolipid synthesis and metabolism; glycerophospholipid synthesis and metabolism pathway; D-glutamine and D-glutamate synthesis and metabolism pathway; and dopamine and L-DOPA synthesis and metabolism pathway.

Samples to be collected include, but are not limited to, tissues samples, such as spinal cord tissue, and blood (serum or plasma), urine, sweat, saliva, tears, spinal fluid, stool, skin, hair, nails as well as other bodily fluids.

Various materials that can be analyzed include, but are not limited to, protein(s), lipid(s), carbohydrate(s), nucleic acids, cellular markers, bacteria and/or chemical moieties. These may be analyzed individually or as a collection. Various patterns to be analyzed include, but are not limited to, genomics, transcriptomics, proteomic, cytometrics, metabolomics and/or ratio(s) and/or combinations of each. Analysis to be performed include, but are not limited to, expression, functional analysis (activity, activation, inhibition, catalysis, resting state, active state, modulation, etc.), quantity, ratio(s), mutation and/or damage. The types of analysis include, but are not limited to, molecular, imaging and psychometric. Analytic methods may include, but are not limited to, immunofluorescence, chemiluminescence, colorimetric, absorbance, electrochemical, acoustic, thermal, colorimetry, radioactive, enzymatic, fluorescent and optical. Analysis outcomes include, but are not limited to, prognostic, diagnostic, predictive, screening, treatment, pharmacodynamic, pharmacokinetic and/or recurrence.

In particular embodiments, indicator or biomarker measurement kit setup can include, but are not limited to, flow cytometer, ELISA, blotting, microarray, gel electrophoresis, test strip, plate assay, RT-PCR, PCR, patch, microscopy, functional, biochemical and biophysical. In further embodiments, the indicator candidates can include, but are not limited to, protein(s), lipid(s), carbohydrate(s), nucleic acids, cellular markers, bacteria and/or chemical moieties related to the synthesis and/or metabolism of tyrosine, tryptophan, linoleic, phenylalanine, sphingolipid, glycerophospholipid, D-glutamine, D-glutamate, dopamine and L-DOPA.

Tyrosine synthesis pathway agents may include, but are not limited to, phenylalanine, phenylalanine hydroxylase, tetrahydrobiopterin, dihydrobiopterin, tyrosine, tyrosine hydroxylase, L-DOPA, Aromatic amino acid decarboxylase, dopamine beta hydroxylase, ascorbate, dehydroascorbate, noradrenaline, S-adenosylmethionine, phenylethanolamine, s-adenosylhomocysteine and adrenaline. Table 1 below provides specific indicators and enzymes as identified in the methods discussed in the experimental data provided below identifying the tyrosine pathway in opioid responsiveness.

TABLE 1
Tyrosine Metabolism Pathway

| Indicators: | Enzymes involved: |
|---|---|
| Tyrosine | 5,6-dihydroxyindole-2-carboxylic acid |
| | oxidase precursor |
| L-dopa | aldehyde dehydrogenase, dimeric NADP- |
| | preferring |
| Dopamine | amine oxidase [flavin-containing] B |
| L-noradrenaline | aromatic-L-amino-acid decarboxylase |
| | isoform/aromatic-L-amino-acid/L- |
| | tryptophan decarboxylase |
| L-adrenaline | catechol O-methyltransferase |
| L-normetanephrine | dopamine beta-hydroxylase |
| L-metanephrine | L-dopachrome tautomerase |
| 3,4-Dihydroxy-phenylacet-aldehyde | membrane primary amine oxidase |
| 3,4-Dihydroxy-phenylacetate | phenylethanolamine N-methyltransferase |
| 3-methoxy-4-hydroxy-phenylglycoaldehyde | thyroid peroxidase precursor |
| 3,4-Dihydroxy-mandelaldehyde | tyrosinase precursor |
| Homovanillate | tyrosine 3-monooxygenase |
| 3,4-Dihydroxy-mandelalate | |
| 3,4-Dihydroxy-phenylethylen-glycol | |
| 5-carboxymethyl-2-hydroxy-muconate | |
| Tyramine | |
| 4-hydroxy-phenylacetaldehyde | |
| 4-hydroxy-phenylacetate | |
| 4-hydroxy-phenylacetylglycine | |
| 4-hydroxy-phenylacetylglutamic acid | |
| 3-Iodo-L-tyrosine | |
| 3,5-Diiodo-L-tyrosine | |
| L-thyroxine | |
| Triiodothyronine | |
| 4-Hydroxy-phenylpyruvate | |
| (R)-3-(4-Hydroxy-phenyl)lactate | |
| Homogentisate | |
| Gentisate-aldehyde | |
| Gentisate | |
| Hydroquinone | |
| 3-Maleyl-pyruvate | |
| 3-Fumaryl-pyruvate | |
| Maleate | |
| Pyruvate | |
| 4-Maleyl-acetoacetate | |
| 4-Fumaryl-acetoacetate | |
| Acetoacetate | |
| dopaquinone | |
| Cysteinyl-DOPA | |
| Dopachrome | |
| Indole-5,6-quinone | |
| 5,6-dihydroxyindole-2-carboxylic acid | |

Trytophan metabolism pathway agents may include, but are not limited to, Clostridium, Lactobacillus, IPA, I3A, indole, tryptophanase, PXR, AhR, GLP-1 and indoxyl sulfate. Table 2 below provides specific indicators and enzymes as identified in the methods discussed in the experimental data provided below identifying the tryptophan pathway in opioid responsiveness.

TABLE 2
Tyrosine Metabolism Pathway

| Indicators: | Enzymes involved: |
|---|---|
| Tryptophan | 3-hydroxyanthranilate 3,4-dioxygenase |
| Tryptamine | acetyl-CoA acetyltransferase |
| N-Hydroxy-tryptamine | acetylserotonin O-methyltransferase |
| Indole | aldehyde dehydrogenase family 7 member A1 |
| Indoxyl | alpha-aminoadipic semialdehyde dehydrogenase |
| Acetylindoxyl | amine oxidase [flavin-containing] B |
| Indole-3-acetaldoxime | aromatic-L-amino-acid decarboxylase isoform/ |
| | aromatic-L-amino-acid/L-tryptophan |
| | decarboxylase |
| Indole-3-acetaldehyde | catalase |
| Indole-3-ethanol | cytochrome P450 1A1 |
| Indolacetate | dihydrolipoamide dehydrogenase |
| N-Formyl-kynurenine | indoleamine 2,3-dioxygenase 1 |
| L-Kynurenine | kynureninase |
| 3-Hydroxy-L-kyurenine | kynurenine formamidase |
| 3-Hydroxy-anthranilate | L-amino-acid oxidase isoform X1 |
| Quinolinate | tryptophan 2,3-dioxygenase |
| Picolinate | |
| 2-Amino-muconate | |
| 2-Oxoadipate | |
| Glutaryl-CoA | |
| Cronyl-CoA | |
| Acetyl-CoA | |
| 5-Hydroxy-L-tryptophan | |
| 5-Hydroxy-formylkynurenine | |
| 5-Hydroxy-kynurenine | |
| serotonin | |
| N-Acetylserotonin | |
| Melatonin | |
| Formyl-N-acetyl-5-methoxykynurenamine | |
| 6-Hydroxymelatonin | |
| N-Methylserotonin | |
| Formyl-5-hydroxy-kynureanmine | |
| 5-Methoxy-tryptamine | |
| 5-Hydroxyindole-acetaldehyde | |
| 5-Hydroxy-indolacetate | |
| 5-Methoxy-indolacetate | |
| Cinnavalininate | |
| Anthranilate | |
| Formyl-anthranilate | |
| 2,3-Dihodrxyindole | |
| Isophenoxazine | |
| gamma-Oxalocrotonate | |

Linoleic acid synthesis and metabolism agents may include, but are not limited to, delta6-desaturase, elongase, delta5-desaturase, arachonic acid, eicosapentaenoic acid, oxidase, docosapenenoic acids, delta12-desaturase, oleic acid, delta15-saturase, gamma-linoenic acid, dihomo-gamma-linolenic acid, stearodonic acid, eicosatetraenoic acid, eicosapentaenoic acid, docosahexanoic acid and stearic acid.

Phenylalanine synthesis and metabolism agents may include, but are not limited to, chorismite, chorismite mutase, prephenate dehydratase, prephenate, prephenate aminotransferase, phenylpyruvate, 4-hydroxyphenylpyruvate, tyrosine aminotransferase, arogenate dehydrogenase, arogenate, tyrosine, phenylalaine, phenylacetyl COA, phenylacetylglutamine, phenylacetate, p-hydroxyphenylpyruvate, homogentisate, maleylacetoacetate, fumarylacetoacetate, fumarate and acetoacetate.

Sphingolipid synthesis and metabolism agents may include, but are not limited to, palmitic acid, palmitoyl CoA, L-serine, serine palmitoyltransfer, 3-keto-dihydrosphingosine, 3-ketodehydrosphingosine reductase, dihydrospingosine, Acyl-CoA, serine palmitoyl transferase, 3-ketosphinganine, 3KS, KDSR, DEGS, dihydroceramide desaturase, ceramide synthase, dihydroceramides, ethanolamine phophate, fatty aldehyde, sphingosine-1-phosphate lyase, Spingosine-1-p, Spingosine kinase, spingosine phosphate, spingosine, ceramidase, dihydroceramide, ceramide, ceramide kinase, ceramide-1-p, phosphatidylcholine, diacylglycerol, sphingomyelin synthase, sphingomyelin, sphingomyelinase, glucosylceramide synthase, galactosylceramide synthase, gangliosides, cerebrosides, lactosylceramides, sulfatides.

Glycerophospholipid synthesis and metabolism agents may include, but are not limited to, glycerol-3-phosphate, acyl-CoA, CoA, 1-acyl-Glycerol-3-phosphate, NADP, NADPH, 1-acyl-DHAP, DHAP, CDP-diacylglycerol, Inositol, CMP, ADP, ATP, diacylglycerol, PI, ethanolamine, phosphoethanolamine, CDP-ethanolamine, choline, phosphocholine, CDP-choline, acyl transferase, acyl transferase, dihydroxyacetone phosphate acyltransferase, acyl-dihydroxyacetone phosphate reductase, phosphatidate cytidylyltransferase, PI synthase, PI-3-kinase, PI-3-P 4-kinase, PI-3,4-P 5-kinase, Type I PI 5-kinase, Type II PI 4-kinase, PI 3-kinase, PA phosphatase, diacylglycerol kinase, ethanolamine kinase, CTP, phosphoethanolamine cytidylyltransferase, CDP-ethanolamine, DAG, phosphoethanolamine transferase, choline kinase, phosphocholine cytidylyltransferase CDP-choline, DAG phosphocholine transferase PE Nmethyltransferase, and 23) PS synthase and PS decarboxylase, cardiolipin, phosphatidylinositol, phosphatidyglycerol, phosphatidylcholine and phosphatidylethanolamine.

D-glutamine and D-glutamate synthesis and metabolism pathway: glutamate, glutamine, GABA, glutaminase, oxoacid, 2-oxoglutarate, glutathione, glucose, arginine, ornithine, glutamate Dh, transaminase arginase INOS, NO, carbamoyl-phosphate synthetase and urea.

Dopamine and L-DOPA Synthesis and Metabolism Pathway: COMT, AADC, MAO-B, 3-O-MethylDopa, DOPAC, 3-metoxy-tyramine, homovanillic acid, amantadine, adrenaline, noradrenaline, l-tyrosine, tyrosinase, l-dopaquinone, melanin, dehydroascorbic acid, ascorbic acid, DOPA decarboxylase, phenylalanine hydroxlase, tyrosinase, tyrosine hydroxylase, DOPAL, CYP2D6, ADH, ALDH, DOPET, 3-methoxytyramine, tyramine, 3-methoxy-4-hydroxyacetaldehyde.

In the methods of the present inventive concept allowing the determination of opioid responsive subjects and opioid non-responsive subjects, the effect of a single treatment or a combination treatment, e.g., change in the level of pain, tolerance, risk of addition or dependence, or efficacy can be quantitated by methods that are well known in the art and as described herein.

In methods of the present inventive concept where is it determined that an opioid and a dopamine modulator such as a D3R agonist should be administered in combination, the opioid and the D3R agonist can be administered in the same composition or formulation and/or in separate compositions or formulations. The separate compositions and/or formulations can be administered simultaneously, concurrently and/or in any order and/or in any interval of minutes, days, weeks, etc. In some embodiments, the D3R agonist may be administered about 1, 2, 3, 4, 5, 10, 15, 20, 25, 30, 35, 40, 45, 50, or 55 minutes or 1, 2, 3, 4, 5, 6, 12, or 18 hours or 1, 2, 3, 4, 5, 6, or 7 or more days before or after administration of the opioid. One or more doses of the D3R against may be administered before or after one or more doses of the opioid.

The type of pain that can be treated according to a method of this invention include neuropathic pain, nociceptive pain, nociplastic pain, mixed pain, central pain, peripheral pain, visceral pain, somatic pain, spinal cord pain, focal pain, multi-focal pain, symmetrical pain, continuous pain, intermittent pain, incident pain, irruptive pain, mild pain, moderate pain, intense pain, traumatic pain, complex regional pain syndrome II, neurological and neuromuscular disease pain, metabolic disease pain, chronic infection pain, cancer pain, genetic pain, Guillain-Barre disease pain, trigeminal neuralgia pain, multiple sclerosis disease pain, fabry's disease pain, HIV/AIDS pain, postherpetic neuralgia pain, neurofibromatosis pain, invasion/compression by tumor pain, postsurgical pain, chemotherapy pain, erythomelalgia pain, paroxysmal extreme pain disorder, fibromyalgia pain, diabetic neuropathy pain, chronic inflammatory pain, allodynia, hyperalgesia, pain from shingles, surgical pain, phantom limb pain, brachial plexus injury, spinal cord injury, nerve lesion, nerve lesion/injury, dental pain, headache, migraine, osteoarthritis, rheumatoid arthritis, post stroke pain, phantom pain, back pain, etc. Pain can be mechanical sensitivity, thermal sensitivity, or other types of pain.

In some embodiments, the pain is post-surgical pain, acute injury pain, chronic pain, cancer pain, dental pain, migraine, back pain, fibromyalgia or gastrointestinal pain.

In particular embodiments, the pain is acute pain, chronic pain, opioid resistant neuropathic pain, or spinal cord injury pain.

Nonlimiting examples of an opioid that can be administered according to a method of the inventive concept include, but are not limited to, generic opioid drugs (e.g., morphine sulfate, fentanyl, methadone hydrochloride, oxymorphone hydrochloride); brand name opioid drugs (e.g., Abstral (fentanyl), Actiq (fentanyl), Avinza (morphine sulfate extended-release capsules), Butrans (buprenorphine transdermal system), Demerol (meperidine [also known as isonipecaine or pethidine]), Dilaudid (hydromorphone [also known as dihydromorphinone]), Dolophine (methadone hydrochloride tablets), Duragesic (fentanyl transdermal system), Fentora (fentanyl), Hysingla (hydrocodone), Methadose (methadone), Morphabond (morphine), Nucynta ER (tapentadol extended-release oral tablets), Onsolis (fentanyl), Oramorph (morphine), Oxaydo (oxycodone), Roxanol-T (morphine), Sublimaze (fentanyl), Xtampza ER (oxycodone), Zohydro ER (hydrocodone)); and combination formulations of opioid drugs (e.g., Anexsia (hydrocodone containing acetaminophen), Co-Gesic (hydrocodone containing acetaminophen), Embeda (morphine sulfate and naltrexone extended-release capsules), Exalgo (hydromorphone hydrochloride extended-release tablets), Hycet (hydrocodone containing acetaminophen), Hycodan (hydrocodone containing homatropine), Hydromet (hydrocodone containing homatropine), Ibudone (hydrocodone containing ibuprofen), Kadian (morphine sulfate extended-release tablets), Liquicet (hydrocodone containing acetaminophen), Lorcet (hydrocodone containing acetaminophen), Lorcet Plus (hydrocodone containing acetaminophen), Lortab (hydrocodone containing acetaminophen), Maxidone (hydrocodone containing acetaminophen), MS Contin (morphine sulfate controlled-release tablets), Norco (hydrocodone containing acetaminophen), Opana ER (oxymorphone hydrochloride extended-release tablets), OxyContin (oxycodone hydrochloride controlled-release tablets), Oxycet (oxycodone containing acetaminophen), Palladone (hydromorphone hydrochloride extended-release capsules), Percocet (oxycodone containing acetaminophen), Percodan (oxycodone containing aspirin), Reprexain (hydrocodone containing ibuprofen), Rezira (hydrocodone containing pseudoephedrine), Roxicet (oxycodone containing acetaminophen), Targiniq ER (oxycodone containing naloxone), TussiCaps (hydrocodone containing chlorpheniramine), Tussionex (hydrocodone containing chlorpheniramine), Tuzistra XR (codeine containing chlorpheniramine), Tylenol #3 and #4 (codeine containing acetaminophen), Vicodin (hydrocodone containing acetaminophen), Vicodin ES (hydrocodone containing acetaminophen), Vicodin HP (hydrocodone containing acetaminophen), Vicoprofen (hydrocodone containing ibuprofen), Vituz (hydrocodone containing chlorpheniramine), Xartemis XR (oxycodone containing acetaminophen), Xodol (hydrocodone containing acetaminophen), Zolvit (hydrocodone containing acetaminophen), Zutripro (hydrocodone containing chlorpheniramine and pseudoephedrine), Zydone (hydrocodone containing acetaminophen)). The opioids of this invention can be employed in the methods of this invention singly or in any combination and/or ratio.

D3R agonists can be any molecule that activates the D3R. In some embodiments, the agonist may specifically activate D3R. In some embodiments, the agonist may predominantly activate D3R over other dopamine receptors. Nonlimiting examples of a dopamine 3 receptor agonist of this invention include enafadotride, cabergoline, PD 128907, pramipexole (Mirapex), pergolide, and rotigotine (Neupro™), singly or in any combination and/or ratio.

It is contemplated that subjects to whom the methods of this invention are applied can receive a lower dose of an opioid when it is administered in combination with a D3R agonist of this invention. By “lower dose” is meant a reduced amount of an opioid relative to the amount that the subject is taking or has been taking without a D3R agonist in combination (e.g., at least about 5%, 10%, 15%, 20%, 25%, 30%, 35%, 40%, 45%, or 50% less). This is because the D3R agonist enhances the efficacy of the opioid, resulting in the same or a better level of pain relief and/or pain control in the subject relative to the amount of pain relief and/or pain control the subject is experiencing in the absence of the D3R agonist.

Thus, in one embodiment, the present invention provides a method for enhancing the therapeutic effect of an opioid in a subject, comprising: a) administering an effective amount of a D3R agonist to a subject; and b) administering a subtherapeutic amount of the opioid to the subject, wherein the therapeutic effect of the subtherapeutic amount of the opioid is enhanced as compared to the effect of the subtherapeutic amount of the opioid administered without the D3R agonist. A “subtherapeutic” amount is an amount of the opioid that does not provide any therapeutic effect when administered on its own, i.e., below the standard dose range for treating acute pain or the standard dose range for treating chronic pain. In some embodiments, a “subtherapeutic amount” is an amount that is less than the amount the subject has been taking to achieve pain reduction and/or pain control in the absence of a D3R agonist (e.g., for chronic pain). In some embodiments, the therapeutic effect is achieved by administering to the subject an effective amount of a dopamine type 3 receptor (D3R) agonist and an opioid to the subject, wherein the D3R agonist allows for a lower dose of the opioid to be administered to achieve pain relief as compared to administration of the lower dose of the opioid without the D3R agonist, thereby treating pain in the subject.

In some embodiments of this invention, a dopamine 1 receptor (DIR) antagonist can be administered in the methods described herein and/or included in the compositions described herein.

In some embodiments, the methods of this invention are carried out without the inclusion of a DIR antagonist and in some embodiments, the compositions do not comprise or include a DIR antagonist. For example, in some embodiments, a composition of this invention can comprise (a) at least one opioid; (b) at least one dopamine type 3 receptor agonist; and (c) a pharmaceutically acceptable carrier, excipient or diluent, with the proviso that the composition does not comprise a DIR antagonist. As another example, in some embodiments, the present invention provides a method of treating pain in a subject in need thereof, comprising administering to the subject an effective amount of an opioid and a dopamine type 3 receptor (D3R) agonist, with the proviso that a DIR antagonist is not administered to the subject.

Nonlimiting examples of a DIR antagonist include ecopipam (SCH 39166), SCH 23390, SKF 83566, singly or in any combination and/or ratio.

It is contemplated that any specific opioid, D3R agonist and/or DIR antagonist, singly or in any combination, can be excluded from the methods and/or compositions of this invention.

In further embodiments, the present invention additionally provides compositions that can be employed in the methods of this invention. Thus, in one embodiment, the present invention provides a composition comprising: (a) at least one opioid; (b) at least one D3 receptor agonist; and (c) a pharmaceutically acceptable carrier, excipient or diluent. The composition can be a pharmaceutical composition or formulation that can comprise additional therapeutic agents or techniques. See, e.g., Remington, The Science And Practice of Pharmacy (latest edition). agents for the treatment and/or management of chronic pain and/or acute pain.

Nonlimiting examples of an additional agent that can be included in a pharmaceutical composition or formulation and/or can be administered according to the methods of this invention include analgesic agents, non-steroid anti-inflammatory (NSAID) agents (e.g., Ibuprofen, Naproxen, Ketoprofen, Diclofenac, Fenoprofen, Ketoroloac, Meloxicam, Indomethacin, Piroxicam, Cox-2 inhibitors, etc.), salicylates (e.g., aspirin, magnesium salicylate, diflunisal, etc.), acetaminophen, codeine, chlorpheniramine, pseudoephedrine, homatropine, triptans, and ergots, in any combination and/or ratio.

In terms of administration of a composition of this invention, the most suitable route in any given case will depend on the nature and severity of the condition and/or the pharmaceutical formulation being administered. The active agents described herein can be formulated for administration in a pharmaceutical carrier in accordance with known practices.

The compositions of the present invention may be suitable for and formulated for parenteral, oral, inhalation spray, topical (i.e., both skin and mucosal surfaces, including airway surfaces), rectal, nasal, buccal (e.g., sub-lingual), vaginal or implanted reservoir administration, etc. where the most suitable route in any given case will depend on the nature and severity of the condition being treated in combination with the drug profile of the compound described herein as would be understood by one of ordinary skill in the art.

For topical administration, suitable forms include, but are not limited to an ointment, cream, emulsion, microemulsion, a gel, a dispersion, a suspension, a foam, an aerosol, a liquid, a droplet, and suitable transdermal delivery systems known in the art, such as patches and bandages, dressing, gauze and the like including the medicament described herein. Topical administration may further include articles of clothing such as socks or hosiery including the medicament described herein.

The term “parenteral” as used herein includes subcutaneous, intradermal, intravenous, intramuscular, intraperitoneal, intra-articular, intra-synovial, intrasternal, intrathecal, intrahepatic, intralesional and intracranial injection or infusion techniques.

Compositions for injection will include the active ingredient together with suitable carriers including propylene glycol-alcohol-water, isotonic water, sterile water for injection (USP), emulPhor™-alcohol-water, cremophor-EL™, polyvinyl pyrrolidone, lecithin, arachis oil or sesame oil, with other additives for aiding solubility or preservation may also be included, or other suitable carriers known to those skilled in the art. Accordingly, these carriers may be used alone or in combination with other conventional solubilizing agents such as ethanol, propylene glycol, or other agents known to those skilled in the art.

Compositions for oral administration may be, for example, solid preparations such as tablets, sugar-coated tablets, hard capsules, soft capsules, granules, powders, gelatins, and the like, with suitable carriers and additives being starches, sugars, binders, diluents, granulating agents, lubricants, disintegrating agents and the like. Because of their ease of use and higher patient compliance, tablets and capsules represent the most advantageous oral dosage forms for many medical conditions.

Similarly, compositions for liquid preparations include solutions, emulsions, dispersions, suspensions, syrups, elixirs, and the like with suitable carriers and additives being water, alcohols, oils, glycols, preservatives, flavoring agents, coloring agents, suspending agents, and the like.

Where the compounds described herein are to be applied in the form of solutions or injections, the compounds may be used by dissolving or suspending in any conventional diluent. The diluents may include, for example, physiological saline, Ringer's solution, an aqueous glucose solution, an aqueous dextrose solution, an alcohol, a fatty acid ester, glycerol, a glycol, an oil derived from plant or animal sources, a paraffin and the like. These preparations may be prepared according to any conventional method known to those skilled in the art.

Compositions for nasal administration may be formulated as aerosols, drops, powders and gels. Aerosol formulations typically comprise a solution or fine suspension of the active ingredient in a physiologically acceptable aqueous or non-aqueous solvent. Such formulations are typically presented in single or multidose quantities in a sterile form in a sealed container. The sealed container can be a cartridge or refill for use with an atomizing device. Alternatively, the sealed container may be a unitary dispensing device such as a single use nasal inhaler, pump atomizer or an aerosol dispenser fitted with a metering valve set to deliver a therapeutically effective amount, which is intended for disposal once the contents have been completely used. When the dosage form comprises an aerosol dispenser, it will contain a propellant such as a compressed gas, air as an example, or an organic propellant including a fluorochlorohydrocarbon or fluorohydrocarbon.

Compositions suitable for buccal or sublingual administration include tablets, lozenges, gelatins, and pastilles, wherein the active ingredient is formulated with a carrier such as sugar and acacia, tragacanth or gelatin and glycerin.

In particular embodiments, the present invention provides a pharmaceutical formulation including the compound described herein wherein the pharmaceutical formulation is a parenteral formulation. In some embodiments, the parenteral formulation is an intravenous formulation. In some embodiments the parenteral formulation is an intraperitoneal formulation. In other embodiments, the present invention provides a pharmaceutical formulation including the compound described herein wherein the pharmaceutical formulation is an oral formulation.

According to the present invention, methods of this invention include administering an effective amount of a composition of the present invention as described above to the subject. The effective amount of the composition, the use of which is in the scope of present invention, will vary somewhat from subject to subject, and will depend upon factors such as the age and condition of the subject and the route of delivery. Such dosages can be determined in accordance with routine pharmacological procedures known to those skilled in the art. A composition of the present invention can comprise the active agents in an amount ranging from a lower limit from about 0.01, 0.05, 0.10, 0.50, 1.0, 5.0, or 10% to an upper limit ranging from about 10, 20, 30, 40, 50, 60, 70, 80, 90, 95, 96, 97, 98, 99, or 100% by weight of the composition. In some embodiments, the active agents include from about 0.05 to about 95% by weight of the composition. In other embodiments, the active agents can include from about 0.05 to about 60% by weight of the composition. In still other embodiments, the active agents include from about 0.05 to about 10% by weight of the composition.

Embodiments of the present invention further provide kits comprising, consisting essentially of or consisting of one or more containers having pharmaceutical dosage units comprising an effective amount of at least one D₁ receptor antagonist, at least one D₃ receptor agonist and optionally an opioid agonist, wherein the container is packaged with optional instructions for the treatment of pain.

The effective dosage of any specific active agent will vary somewhat from composition to composition, patient to patient, and will depend upon the condition of the patient and the route of delivery. As a general proposition, a dosage from about 0.1 to about 50 mg/kg will have therapeutic efficacy, with still higher dosages potentially being employed for oral administration, wherein aerosol administration is usually lower than oral or intravenous administration. Toxicity concerns at the higher level may restrict intravenous dosages to a lower level such as up to about 10 mg/kg, all weights being calculated based upon the weight of the active base, including the cases where a salt is employed. For the opioid component, a typical dose may range from about 0.1 mg/day to about 6 mg/day for intravenous or intramuscular administration. A dosage from about 0.1 mg/day to about 60 mg/day may be employed for oral administration. The D3R agonist dose may range from about 0.05 mg/day to about 50 mg/day.

In particular embodiments, administration to a subject such as a human, a dosage of from about 0.1 mg/day (e.g., about 1.0 mg/day), up to about 60 mg/day for opioid and from about 0.05 mg/day to about 100 mg/day (e.g., about 50 mg/day) for D3R agonist or more can be employed. Depending on the solubility of the particular formulation of active agents administered, the dose, which in some embodiments, can be in hourly (e.g., every four hours; every six hours; every 12 hours, etc.), daily (e.g., once a day; twice a day, etc.), weekly (e.g., once a week; twice a week; four times a week, etc.), monthly and/or yearly increments, can be divided among one or several unit dose administrations.

### Definitions

As used herein, “a” or “an” or “the” can mean one or more than one. Also as used herein, “and/or” refers to and encompasses any and all possible combinations of one or more of the associated listed items, as well as the lack of combinations when interpreted in the alternative (“or”).

Furthermore, the term “about,” as used herein when referring to a measurable value such as an amount of a compound or agent of this invention, dose, time, temperature, and the like, is meant to encompass variations of 20%, 10%, 5%, 1%, 0.5%, or even 0.1% of the specified amount.

The terms “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

The term “modulate,” “modulates” or “modulation” refers to enhancement (e.g., an increase) or inhibition (e.g., a reduction) in the specified activity.

The term “management” as used herein refers to the ability to affect a method, process, state of being, disorder or the like. The effect may be that of prevention, treatment or modulation.

By the terms “treat,” “treating” or “treatment of,” it is intended that the severity of the disorder or the symptoms of the disorder are reduced, or the disorder is partially or entirely eliminated, as compared to that which would occur in the absence of treatment. Treatment does not require the achievement of a complete cure of the disorder.

By the terms “preventing” or “prevention,” it is intended that the inventive methods eliminate or reduce the incidence or onset of the disorder, as compared to that which would occur in the absence of the measure taken. Alternatively stated, the present methods slow, delay, control, or decrease the likelihood or probability of the disorder in the subject, as compared to that which would occur in the absence of the measure taken.

A “therapeutically effective” or “effective” amount is intended to designate a dose that causes a relief of symptoms of a disease or disorder as noted through clinical testing and evaluation, patient observation, and/or the like. “Effective amount” or “effective” can further designate a dose that causes a detectable change in biological or chemical activity. The detectable changes may be detected and/or further quantified by one skilled in the art for the relevant mechanism or process. Moreover, “effective amount” or “effective” can designate an amount that maintains a desired physiological state, i.e., reduces or prevents significant decline and/or promotes improvement in the condition of interest. As is generally understood in the art, the dosage will vary depending on the administration routes, symptoms and body weight of the patient but also depending upon the compound being administered.

“Tolerance” refers to a declining response to treatment over time.

“Drug tolerance” means a decreasing response to repeated constant doses of a drug or the need for increasing doses to maintain a constant response.

“Withdrawal” refers to a group of symptoms that occur upon the abrupt discontinuation or decrease in intake of medications or recreational drugs.

“Antinociceptive” refers to any factor that increases tolerance for, or reduces sensitivity to, a dangerous or harmful stimulus, i.e., a stimulus that causes pain.

“Dependence” refers to the need for one or more substances (i.e., drugs) to function. It is possible to be dependent on a drug and not be addicted. Dependence can be a bodily response to a substance. Dependence often occurs when a subject relies on medications to control a chronic medical condition.

D3R is dopamine 3 receptor agonist.

PRAM is pramipexole, a clinically available dopamine 3 receptor agonist with preference for dopamine 3 receptors.

Morph is morphine, which is an opioid prescribed for pain.

Oxycodone is a semi-synthetic opioid prescribed for pain.

“In combination with” means sufficiently close in time to produce a combined effect (that is, in combination with can be simultaneously, or it can be two or more events occurring within a short time period before or after each other). In some embodiments, the administration of two or more compounds in combination with means that the two compounds are administered closely enough in time that the presence of one alters the biological effects of the other. The two compounds can be administered in the same or different formulations or sequentially. Such concurrent administration can be carried out by mixing the compounds prior to administration, or by administering the compounds in two different formulations, for example, at the same point in time but at different anatomic sites or using different routes of administration.

“Chronic pain” is pain that extends beyond the expected period of healing, and may be considered pain that has lasted for at least three months.

A “subject” as used herein can be a human subject and can include, but is not limited to, a patient. The subject may be male or female and may be of any race or ethnicity, including, but not limited to, Caucasian, African-American, African, Asian, Hispanic, Indian, etc. The subject may be of any age, including newborn, neonate, infant, child, juvenile, adolescent, adult, and geriatric. A subject can also include an animal subject, including mammalian subjects such as canines, felines, bovines, caprines, equines, ovines, porcines, rodents (e.g., rats and mice), lagomorphs, primates (including non-human primates), etc., for prevention and treatment purposes as well as veterinary medicine and/or pharmaceutical drug development purposes. A subject of this invention can be experiencing pain and/or are at increased risk of experiencing pain, e.g., as a result of surgery and/or a medical procedure. In some embodiments, the subject has experienced a spinal cord injury. In some embodiments, the subject is classified as responsive to opioid therapy. In other embodiments, the subject is classified as non-responsive to opioid treatment, e.g., de minimis to no pain relief when treated with opioid medications.

Having now described the invention, the same will be illustrated with reference to certain examples, which are included herein for illustration purposes only, and which are not intended to be limiting of the invention.

### Examples

### Animals

All experimental procedures complied with the National Institute of Health (NIH) guidelines for animal care and were approved by the East Carolina University Institutional Animal Care and Use Committee. Female, Long-Evans rats (weighing 200-225 g;) were purchased from Charles River Laboratories (Wilmington, MA). Females are specifically chosen because they have fewer and less severe urological complications after the contusion injury used. Animals were maintained on a 12-h light-dark cycle in a temperature-controlled environment with food and water available continuously. Rats were housed in standard acrylic rat cages with wood shavings for bedding and a PVC tube for enrichment. Animals were kept in pairs prior to surgery and individually afterwards.

### Contusion SCI Model

As previously described, a contusion model of SCI was used (Rodgers et al 2019). Generally, a minimum of 8 animals are needed to detect a significant difference in thermal threshold under varying drug conditions, and only ˜⅓ of animals are responsive to morphine after SCI (Rodgers, Yow, et. al., 2019). Therefore, to generate enough morphine-responsive animals for comparison to non-responders on behavioral assessments, we produced 30 SCI animals to obtain 10 morphine responders and for comparison to non-responders in this study.

Briefly, 30 rats were anesthetized with inhaled Isoflurane (1-3% to effect) and placed in a stereotactic apparatus (Stoelting Co, Wood Dale, IL). The vertebral column was exposed, and a spinous process and vertebral lamina removed from one spinal level between T12-L1. A contusion injury (velocity of impact=0.8 m/see, depth of impact=1.5 mm, dwell time=85 ms) was produced 0.5 mm to the right of midline using a microprocessor-controlled impactor (PCI3000 Precision Impactor, Hatteras Instruments, Cary, NC) with a custom-designed 1 mm round, blunt impact tip. Muscle and fascia were closed with absorbable sutures (chromic catgut, DemeTech, Miami Lakes, FL) and skin was closed using staples (Appose ULC AutoSuture Slim Body Skin Stapler, Covidien, Mansfield, MA). Topical anesthetic was applied to the incision immediately after surgery. All surgical procedures were completed in 20-40 minutes. The above parameters result in a mild-moderate injury (Tartar et al., 2009), which can cause transient loss of bladder function and hind limb paralysis in some animals. In those animals, bladders were expressed manually twice daily until function was recovered (a period that lasted up to 4 days).

### Assessment of Locomotor Recovery

Following SCI, animals were assessed weekly prior to nociceptive testing, for hindlimb motor function using Basso, Beattie, Bresnahan (BBB) Locomotor Scale (Basso et al., 1995). Briefly, animals were placed in an open field within the laboratory and observed by two investigators for 4 minutes (performed between 1400-1600 h). The BBB scale assesses hindlimb movements (at 3 joints), weight-bearing ability coordination, toe clearance, and trunk stability, with scores ranging from 0-21. Only animals that scored 15 points or higher underwent nociceptive testing to ensure that motor impairment was not contributing to changes in nociceptive thresholds.

### Nociceptive Testing

To test spinal cord-mediated reflexes, animals were tested during mid photo-phase (approximately 0900-1400 h) to minimize potential circadian effects. All animals were habituated over a minimum of 5 days prior to testing. All behavior testing was performed by the same experimenter who was blinded to pharmacological treatment condition. Thermal nociception was measured using tail flick latency (TF-1; Columbus Instruments, Columbus OH). Testing followed methods previously described (Rodgers, Yow et al. 2019). Briefly, for each test, the rat was gently restrained, and its tail was placed over the analgesia meter window approximately 4 cm from the tip. a cutoff latency of 10 s was used to prevent possible tissue damage. Average tail-flick latency was calculated over 3 trials.

### Pharmacological Treatment

To assess morphine responsiveness and the role of dopamine receptor modulators after SCI we used: morphine (2 mg/kg; Morphine sulfate salt pentahydrate, M8777, Sigma-Aldrich, St. Louis, MO, USA), saline (volume matched to morphine injection; 0.9% Sodium Chloride Injection, USP; NDCN 0409-4888-02; Hospira, Lake Forest, IL), the dopamine D₃ receptor agonist, pramipexole (0.1 mg/kg; A1237, Sigma-Aldrich) and the dopamine D₁ receptor antagonist, SCH 39166 (0.1 mg/kg; 2299; Tocris Bioscience, Minneapolis, MN). All drugs were freshly prepared before use; morphine and pramipexole were dissolved in sterile saline (0.9% NaCl) (NDC 0409-4887-17; Hospira) and SCH 39166 was dissolved in 1% dimethyl sulfoxide (DMSO, 472301, Sigma-Aldrich) solution in sterile saline.

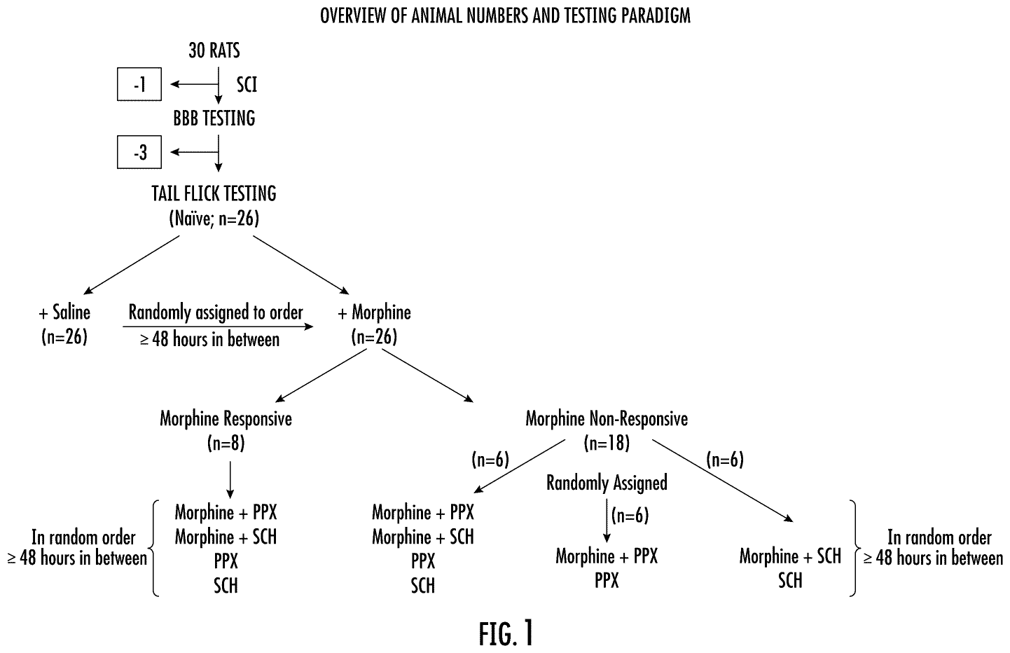

All drugs were administered via subcutaneous (s.c.) injection 30 mins prior to behavioral testing by an investigator not involved in the behavioral testing. Post-injury testing began at 21 days post-surgery when all animals were tested as drug naïve before testing under the different drug conditions. All testing sessions were separated by a minimum of 48 hours. Animals were first tested under saline and morphine drug conditions. Half of the animals were randomly assigned using a random number generator to receive saline first, whereas the other half received morphine first and 48 hours later were tested on the opposite drug condition. Based on the results, animals were divided into morphine responsive vs. morphine non-responsive groups. Animals were categorized as being morphine-responsive if their thermal thresholds were statistically significantly increased (p<0.05) with an acute injection of morphine compared to injection of saline. Non-responsive animals are those whose thermal thresholds did not increase significantly with an acute injection of morphine compared to injection of saline. The morphine non-responsive animals were subdivided such that ⅓ animals were tested under all drug conditions, ⅓ were exposed to only the DIR antagonist and ⅓ only exposed to the D3R agonist. This subdivision was done using matched pairing of morphine responsiveness and animals were then randomly assigned to either the DIR antagonist group or the D3R agonist group. (FIG. 1).

### Vaginal Cytology Assessment

Previous studies have demonstrated that both baseline pain thresholds and antinociceptive response to morphine is impacted by estrus stage in female rats, with proestrus and estrus being associated with increased pain sensitivity and morphine tolerance being enhanced during proestrus. (Shekunova and Bespalov 2004, Terner, Lomas et al. 2005, Ibironke and Aji 2011). Ovariectomy of female rats decreased pain sensitivity and restored the analgesic potency of morphine (Shekunova and Bespalov 2004, Terner, Lomas et al. 2005). Therefore, to include the potential impact of estrus in our studies, we also assessed the estrus stage of our rats on the day of nociceptive testing. To determine stage of estrus in SCI rats, vaginal smears were performed for cytological assessment on a separate group of SCI rats (n=14) immediately following tail flick testing. Rats were gently restrained, and 0.2 ml of sterile saline was introduced directly into the vaginal canal using a 1 ml syringe without a needle. The saline was carefully aspirated back into the syringe, then reintroduced and aspirated for a total of 3-4 flushes. The fluid was withdrawn, a drop was placed on a glass slide, and the edge of a second slide was used to make a smear with a feathered edge. The slides were allowed to air dry. Once dry, slides were processed using a Wright-Giemsa stain using a Quick Slide Plus II automatic slide processor. Slides were assessed by two independent raters who were blinded as to behavioral grouping of each rat (i.e., responder or non-responder), when raters did not agree a third independent rater was used to determine classification.

### Tissue Collection and Analysis

At the conclusion of behavioral testing, approximately 5 days after the last drug condition, animals were deeply anesthetized and decapitated. The brain was removed from the skull and the striatum (both nucleus accumbens and dorsal striatum) from both hemispheres was dissected out for mass spectrometry. Three sections of spinal cord were collected: the area around injury for lesion analysis and two sections of lumbar cord below the level of injury for mass spectrometry. Samples for mass spectrometry were flash frozen in liquid nitrogen. The same tissues were extracted from 4 uninjured animals to serve as controls.

### Lesion Analysis

To ensure that any differences in behavior were not attributable to differences in lesion volume, injured segments of spinal cord were placed in 4% paraformaldehyde for 24 hours followed by 30% sucrose for at least 48 hours. Using a Leica 2400 freezing microtome, serial 75 μm longitudinal sections were cut through the 0.5 cm length of cord surrounding the lesion site, collected in PBS, mounted on TruBond 380 adhesive microscope slides (#0380G, Matsunami, Bellingham, WA) and stained with cresyl violet.

Sections from randomly selected animals (n=5 morphine responders; n=8 morphine non-responders) were then imaged on an Olympus BX51 microscope with an Olympus DP70 camera. For each animal, the section of cord with the largest lesion area was used to quantify the extent of gray matter damage.

Using Image J (NIH) after setting the scale, the max width and length of the lesion was measured in millimeters (mm), those measurements were then multiplied to get an approximate area of the lesion. The number of sections containing the lesion were also counted as a secondary measure. Lesion analysis was performed by an investigator blinded to the behavioral group of each animal.

### Mass Spectrometry/Metabolomics

Mass spectrometry and metabolomics were performed on 5 randomly selected animals from each group (responsive vs. nonresponsive). For extraction, below-level lumbar cord and striatum samples were weighed and transferred to centrifuge tubes containing silica beads, LC-MS grade Methanol and dopamine-d₃ (internal standard). Samples were homogenized using a bead mill homogenizer for 10 seconds and then sonicated for 60 seconds. The samples were subsequently centrifuged at 12 000 rpm for 10 mins at 4° C. The clarified supernatant was collected and stored at −80° C. until further processing. Prior to analysis, samples were dried to completeness with ultrapure N₂ and resuspended in 50:50 methanol:water with 0.1% formic acid.

### Untargeted Metabolomics LC/MS

An Eksigent 425 microLC/SCIEX 5600+ triple time-of-flight mass spectrometer was used to conduct untargeted metabolomics analysis. A Halo C18 0.5×50 mm 2.7 μm column was used for separation of the analytes using micro flow LC. The flow rate was 10 μl/min and 5 μL of sample was injected. Mobile phase A consisted of water with 0.1% formic acid and mobile phase B was acetonitrile. A linear gradient was utilized starting with 10% B for 2 min, increasing to 90% B for 15 min, held for 5 min, decreased back to 10% B over 2 min and equilibrated for 10 min for a total run time of 30 min.

Data was acquired for MS and tandem MS/MS analysis using independent data acquisition for the top 20 most abundant ions in positive ionization mode. The scan range for MS was 80-1250 Da. Principal component analysis (PCA) and t-tests were conducted using MarkerView 1.3.1. Putative metabolite/pathway identification was performed by using HMDB.ca database (molecular weight tolerance±0.05 DA, Adduct type: M+H, and M+Na) to identify peaks. MetaboAnalyst (www.metaboanalyst.ca) was utilized to conduct pathway analysis (Fisher's Exact Test and relative-betweenness centrality, Rattus norvegicus pathway library).

### Targeted LC/MS

Targeted LC/MS was used to quantify the amount of dopamine present in the striatum and spinal cords of morphine responsive, morphine non-responsive and uninjured naïve animals. A ThermoSci hypersil gold 50×3 mm column was used for separation of the analytes on an Exion HPLC. The column temperature was maintained at 32° C. A gradient was used to separate the compounds using mobile phase A: water with 0.1% formic acid and mobile phase B: methanol. A linear gradient was performed as follows: 0% B from 0 to 1 min, ramp to 97% B to 8 min, hold at 97% B from 8 to 9 min, 0% B from 10-15 min (15 min total run time at a flow rate of 0.3 mL/min) for each 20 μL sample injection. Multiple reaction monitoring MS-MS analysis was conducted on an AB SCIEX 3200 triple quadrupole mass spectrometer in positive ionization mode (SCIEX Analyst). The source parameters were the following: curtain gas 50 psi; heater gas 50 psi, ion spray voltage 5500 V; source temperature 500° C. Parameters were optimized using direct infusion of each analyte using a split tee injection with the LC flow. Quantitative analysis was performed using MultiQuant using deuterated dopamine as an internal standard (Parent ion mass: 152.100 Da; fragment ion masses: 137.100 and 91.100 Da). Least squared regression with 1/x weighing was used to evaluate the linearity of the response with adequate compensation for heteroscedasticity during all experiments.

### Statistical Analysis

Tail flick results are presented as percent analgesia, which is calculated as maximum possible effect (MPE) using the following formula MPE=[(test latency−baseline latency)/(cutoff latency-baseline latency)]*100. A one-way ANOVA was used to assess effect of drug condition. Holm-Sidak multiple comparison test was used to compare morphine alone to each drug condition. Lesion data was analyzed using unpaired t-tests. Metabolomics and targeted mass spectrometry analyses are described above. Data are presented as means±SEM. Differences at p<0.05 were considered significant. Analyses were done using Prism 7 v. 7.02 (GraphPad Software, Inc, LaJolla, CA).

### Results

SCI animals show a dichotomous response to morphine. Following surgery one rat was lost due to complications. To assess hindlimb motor functioning in the remaining animals, BBB testing was performed weekly after surgery. On week 1, SCI animals showed moderate-mild motor impairments (BBB scores ranged from 13-21; mean=19.86±0.44) that gradually resolved, and by two weeks post-injury, motor functions had returned to normal in almost all SCI animals (range=14-21; mean=20.36±0.36). At that time, three rats were excluded from the study based on their failure to meet the criteria of a BBB score of at least 15 at three weeks post-injury. Therefore, of the 30 SCI animals, 26 animals were used for drug testing.

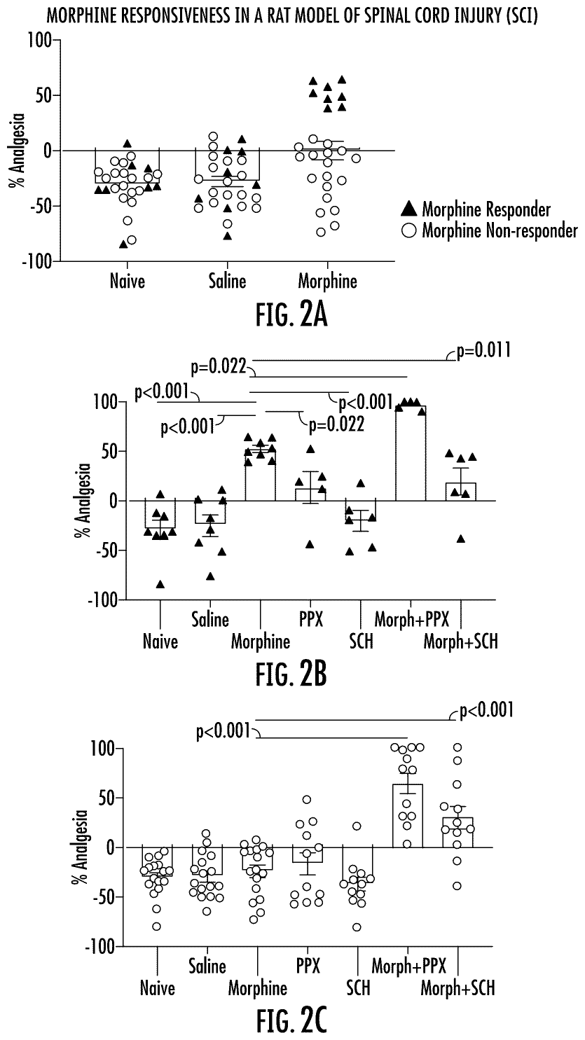

Thermal nociceptive thresholds were examined to assess if SCI animals developed the previously reported pain condition of hyperalgesia (Vierck, Siddall et al. 2000, Kerr and David 2007, Sharp, Boroujerdi et al. 2012, Rodgers, Yow et al. 2019) and their responsiveness to a single acute dose of morphine (2 mg/kg) was determined. The mild contusion injury induced thermal hyperalgesia as evidenced by a significant reduction in percent analgesia of −30.14%±4.106 and −27.85%±4.702 in the post-injury drug naïve and saline conditions respectively, compared to a baseline latency of 5.4±0.13 seconds (FIG. 2A). An acute injection of morphine resulted in a dichotomous response, providing analgesia in one third of SCI animals only (n=8); the average % analgesia of morphine responsive SCI rats was 52.01%±3.494. The remaining two thirds of SCI animals (n=18) were nonresponsive to morphine and showed % analgesia scores (−24.48%±6.241) similar to the drug naïve and saline conditions (−30.92%±4.705 and −29.91%±5.298, respectively). Response to morphine was not dependent on level of hyperalgesia experienced post injury as Pearson's correlation coefficient failed to show a significant correlation between drug naive and morphine % analgesia scores (r(24)=0.23; p=0.26).

D3R agonist or DIR antagonist combined with morphine has differential effects dependent on morphine responsiveness. It was demonstrated that a dopamine D3R agonist or a DIR antagonist could restore the morphine response in SCI rats that were nonresponsive to morphine (Rodgers, Yow et al. 2019). Here, the differences between morphine-responsive and nonresponsive SCI animals to dopamine modulators in combination with morphine and alone were determined.

In morphine responsive animals (n=8), comparison of pharmacological conditions showed a significant effect [F (6,39)=20.54, p<0.0001] (FIG. 2B). Holm-Sidak multiple comparison testing revealed that the addition of pramipexole to morphine, like in nonresponsive animals, significantly increased % analgesia (96.98%±1.985) compared to morphine alone (52.01%±3.494). However, the addition of a DIR antagonist, SCH39166, had the opposite effect; % analgesia significantly decreased (18.79%±13.547) compared to morphine alone condition (52.01%±3.494) in morphine responsive animals. In the morphine non-responsive animals, injection of either pramipexole or SCH 39166 alone significantly decreased % Analgesia scores (12.87%±15.84 and −21.07%±10.46, respectively) compared to morphine alone (52.01%±3.494; p=0.0215 and p<0.0001, respectively).

In morphine non-responsive animals (n=18), comparison of pharmacological condition on thermal thresholds showed a significant effect [F (6, 92)=22.03; p<0.0001]. Consistent with previous results, Holm-Sidak multiple comparison testing showed that the addition of a D3R agonist, pramipexole, to morphine significantly increased % Analgesia from −24.48%±6.241 to 64.38%±10.37 (p<0.0001) (FIG. 2C). Likewise, addition of a DIR antagonist, SCH 39166, to morphine increased % analgesia from −24.48%±6.241 to 29.56%+11.51 (p<0.0001). Injection of pramipexole or SCH 39166 alone did not significantly increase % Analgesia (−7.27%±11.06 and −37.5%±6.99, respectively) compared to morphine alone condition −24.48%±6.241, (p=0.8854 and p=0.6511, respectively).

Lack of morphine responsiveness is not associated with stage of estrus in female SCI rats. To determine if morphine responsiveness was dependent on stage of estrus, in a separate group of SCI female rats (n=14), vaginal smears were taken immediately following tail flick testing with an acute dose of morphine (2 mg/kg, SC). Cytological assessments show that nonresponsive rats (n=10) spanned all stages of estrus with 3 in proestrus, 4 in estrus, 1 in metestrus and 2 in diestrus. Of the 4 morphine responsive rats, 3 were in proestrus and 1 in estrous. This supports that lack of morphine responsiveness was not dependent on estrus cycle phase.

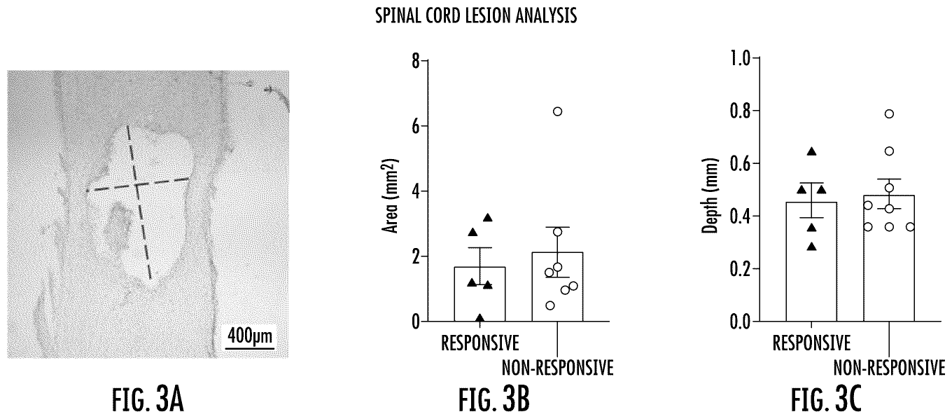

Morphine response is independent of size of spinal cord lesion in SCI animals. Next, we assessed whether lesion size was affecting morphine responsiveness. Lesions in both groups of animals showed cystic cavitation and loss of neural tissue. Longitudinal sections were stained with cresyl violet and we measured the length and width of the epicenter of each lesion to provide an estimated area for the lesion (FIG. 3A). Calculated lesion areas were similar between morphine responsive (n=5) (1.676 mm²±0.5621) and nonresponsive (n=8) rats (2.103 mm²±0.7695; t=0.4159, p=0.6863; FIG. 3B). As a secondary measure of lesion size, we also assessed depth to the epicenter of each lesion. Depth of lesion for morphine responsive rats (0.480 mm±0.065) was not significantly different compared to nonresponsive rats (0.5075 μm±0.058; t=0.3063; p=0.7651; FIG. 3C).

### Metabolomics

An untargeted metabolomics approach was used to investigate the neurochemical consequences of SCI and to explore differences between injured animals that are morphine responsive versus nonresponsive.

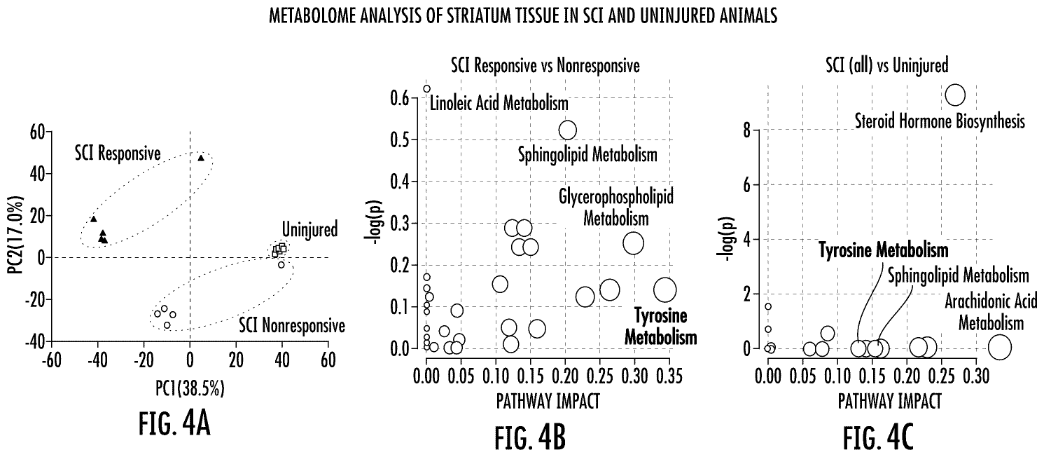

These neurochemical differences in the striatum (both nucleus accumbens and dorsal striatum) and in lumbar spinal cord below the level of injury in 5 morphine responders, 5 morphine non-responders and 5 uninjured naïve animals were assessed. FIG. 4A shows the scores plot for the PCA analysis highlighting in the striatum three sample clusters, the overview of metabolic pathways from metabolites significantly different (p-value<0.05) between SCI to the responsive and nonresponsive morphine groups and the SCI to the uninjured groups, and the tyrosine metabolite pathway demonstrating which metabolite(s) were significantly different in the comparisons. PCA identified three distinct clusters that corresponded to our three groups (uninjured animals, SCI morphine nonresponsive animals, and SCI morphine responsive animals). Principal component 1 accounted for 38.5% of the variation between the three groups and component 2 accounted for 17% of the variation between groups. Next, using a t-test peaks that were significantly different with a p-value<0.05 between SCI morphine responsive versus nonresponsive animals were tentatively identified. A total of 72 peaks were revealed to differ between these animals. For putative metabolite/pathway identification, we used the HMDB.ca database (molecular weight tolerance±0.05 DA, Adduct type: M+H, and M+Na) to identify peaks. Potential metabolites were then entered into MetaboAnalyst.ca to conduct pathway analysis (Fisher's Exact Test and relative-betweenness centrality, Rattus norvegicus pathway library). The results of the pathway analysis are shown in a graphical visualization provided by MetaboAnalyst (FIG. 4B). Tyrosine metabolism pathway was shown as having a high impact, and the putatively identified components included L-DOPA and dopamine. Other pathways with substantial changes are labeled within the figure and include linoleic acid metabolism, sphingolipid metabolism and glycerophospholipid metabolism.

Next, changes as a result of injury were examined by assessing differences between all 10 SCI animals (regardless of morphine responsiveness) versus 5 uninjured animals. Using a t-test, a total of 135 peaks that differed significantly were identified. Pathway analysis in MetaboAnalyst (FIG. 4C) identified multiple pathways, again including tyrosine metabolism, specifically identifying dopamine. Other identified pathways again included sphingolipid metabolism as well as steroid hormone biosynthesis and arachidonic acid metabolism.

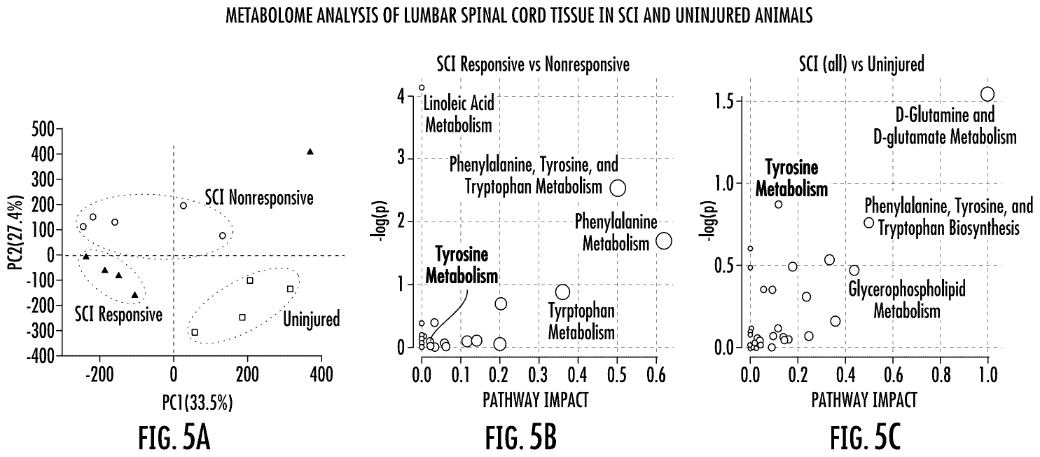

Using the same untargeted metabolomics approach, the SCI-induced changes in metabolomics below the level of injury in the lumbar spinal cord were assessed. FIG. 5 shows the spinal cord PCA results and resulting pathway analysis. As with the striatum, PCA identified three distinct clusters that corresponded to our three groups (uninjured animals, SCI morphine nonresponsive animals, and SCI morphine responsive animals). Principal component 1 accounted for 33.5% of the variation between the three groups and component 2 accounted for 27.4% of the variation between groups. Next, using a t-test peaks that significantly differed between SCI morphine responsive versus nonresponsive animals were tentatively identified. A total of 39 peaks were revealed to differ between these animals. For putative metabolite/pathway identification, we used the HMDB.ca database to identify peaks. Potential metabolites were then entered into MetaboAnalyst.ca to conduct pathway analysis. The results of the pathway analysis are shown in the graphical visualization provided by MetaboAnalyst (FIG. 5B).

Pathways that showed high significance/impact include tyrosine and tryptophan biosynthesis, tryptophan metabolism, linoleic acid metabolism and phenylalanine metabolism. Tyrosine metabolism was identified in pathway analysis but with lower impact/significance compared to the striatal data. Next, neurochemical changes in the spinal cord as a result of injury were examined by assessing differences between all SCI animals (regardless of morphine responsiveness) and uninjured animals.

Using a t-test, a total of 84 peaks that significantly differed were identified. Pathway analysis in MetaboAnalyst (FIG. 5C) identified multiple pathways, including tyrosine metabolism. Other identified pathways included D-glutamine and D-glutamate metabolism, phenylalanine, tyrosine, and tryptophan biosynthesis, and glycerophospholipid metabolism.

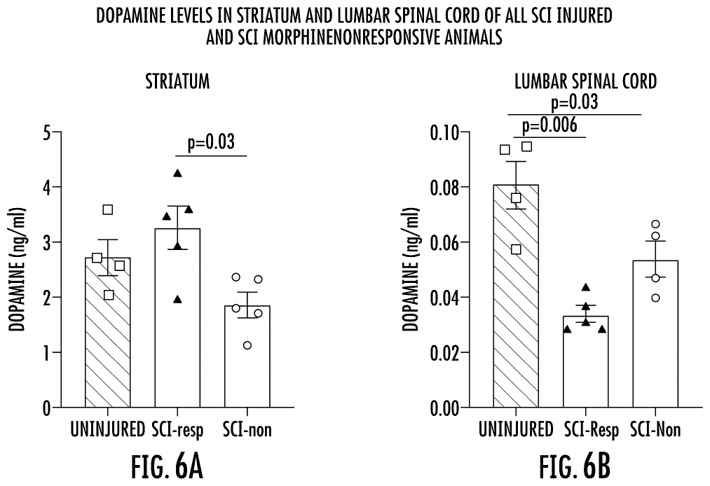

In SCI animals, dopamine levels are altered in the striatum and lumbar spinal cord. Since dopamine and the tyrosine metabolism pathway were putatively identified in the metabolomics analysis, dopamine levels in the striatum and lumbar spinal cord of uninjured and SCI animals using targeted mass spectrometry were assessed. In the striatum, a one-way ANOVA showed a significant effect for group [F (2,11)=5.189, p=0.0259]. Holm-Sidak multiple comparison testing showed a significant difference between SCI animals depending on morphine response (FIG. 6A). SCI animals that were nonresponsive to morphine had significantly lower dopamine levels (1.86±0.23 ng/ml), in the striatum, compared to morphine responsive SCI animals (3.245±0.38 ng/ml; p=0.0253). No significant differences were seen in dopamine levels between uninjured animals (2.717±0.32 ng/ml) and morphine responsive SCI animals (3.245±0.38 ng/ml; p=0.2753) nor uninjured animals (2.717±0.32 ng/ml) and morphine nonresponsive SCI animals (1.86±0.23 ng/ml; p=0.1702). Assessing dopamine levels in lumbar spinal cord, a one-way ANOVA showed a significant effect for group [F (2,10)=15.16, p=0.0008]. Holm-Sidak multiple comparison testing showed a significant difference between uninjured animals and SCI animals (FIG. 6B). SCI animals that were responsive to morphine had significantly less dopamine (0.039±0.044 ng/ml) in the spinal cord than uninjured animals (0.08±0.009 ng/ml; p=0.0006). Likewise, SCI animals that were nonresponsive to morphine also had significantly less dopamine (0.054±0.006 ng/ml) than uninjured animals (0.08±0.009 ng/ml; p=0.0315). There was no significant difference in dopamine levels between SCI morphine responsive animals (0.039±0.044 ng/ml) and nonresponsive animals (0.054±0.006 ng/ml; p=0.087).

### DISCUSSION

While opioids are not the first choice for treating SCI-related pain, they continue to be used (Wong, Alexander et al. 2019) despite the fact that they are not universally effective and have inherent safety concerns. As reported in humans (Attal, Guirimand et al. 2002), this animal model of SCI confirms that only ⅓ of injured animals experienced significant analgesia when treated with opioids after injury. In these morphine-responsive animals, the combination of morphine with the D3R agonist further, and significantly, increased the analgesic response. Importantly, in morphine nonresponsive animals, the combination approach was similarly effective. As neither morphine nor pramipexole bind to the DIR receptor (Millan, Maiofiss et al. 2002, Chen, Collins et al. 2008) but both receptor pathways decrease adenylate cyclase (AC) activity, their synergistic action after spinal cord injury might derive from independently decreasing cAMP-mediated downstream effects. In support of this synergistic model, the analgesic response to the morphine/pramipexole combination in morphine-unresponsive animals, while still significantly increased, is smaller than that seen in the morphine-responsive animals.

In the striatum, SCI responders vs. SCI non-responders had distinct profiles with regards to the DA metabolic pathway, including pathways associated with tyrosine metabolism which is critical for dopamine production. Mass spectrometry confirmed that morphine responsive animals had significantly higher levels of dopamine in the striatum than did morphine nonresponsive animals. The ventral striatum, specifically the nucleus accumbens (NAc), receives pain inputs directly from the spinal cord (Burstein and Giesler 1989) as well as from other pain-related brain structures and this region plays a role in pain processing as well as opioid regulation (Harris and Peng 2020). Stimulation of DA in the striatum results in antinociception and D2R agonists delivered to the NAc can block nociception in the formalin test in rodents (Harris and Peng 2020). There is also clear evidence that the mesolimbic dopamine system is altered by the presence of pain (Wawrzczak-Bargiela, Ziolkowska et al. 2020) and can control the efficacy of opioid pain medications. Studies have shown that the analgesic effects of morphine can be abolished by blocking dopamine receptors in the NAc (Mitsi and Zachariou 2016).

In the spinal cord tissue, tyrosine metabolism was associated with injury itself regardless of morphine responsiveness and mass spectrometry demonstrated that dopamine levels were significantly decreased in both responders and non-responders compared to uninjured controls. Studies have reported conflicting results of decreased dopamine levels and the enzymes involved in DA synthesis in the spinal cord (Voulalas, Ji et al. 2017) and at the same time, increases in DA production in the cord after injury (Weaver, Cassam et al. 1997, Hou, Carson et al. 2016). Morphine responders and non-responders did differ with respect to tyrosine biosynthesis, a finding not seen in the striatum.

Outside of the dopamine system, sphingolipid metabolites also differed between opioid responders vs. non-responders. Sphingolipids have long been studied with regards to their effect on secondary injury as well as recovery from SCI and are more recently being examined as a target for SCI therapy (Jones and Ren 2016). Our results confirm that SCI affects the pathways associated with sphingolipid metabolism. Sphingolipids have not only been linked to the development of pain (Squillace, Spiegel et al. 2020), but these bioactive lipids also have been shown to prevent the analgesic actions of opioids (Muscoli, Doyle et al. 2010, Doyle, Janes et al. 2020).

Compared to conventional methods that attempt to assess the likelihood of success of opioid therapy, the present inventive concept provides methods and tools for determining opioid responsiveness and overcoming opioid desensitization in pain including, but not limited to, acute pain, chronic pain, opioid resistant neuropathic pain, and spinal cord injury pain, by identifying the metabolomic profile of the dopaminergic system in opioid responsive and opioid non-responsive subjects. Thus, the present inventive concept allows for successful methods for treating and/or managing pain. The inventive concept also provides methods for reducing or inhibiting opioid tolerance, reducing risk of opioid addiction or dependence, restoring opioid efficacy, and enhancing the therapeutic effect of an opioid.

The current approach further provides advantages of being non-invasive, responsive to the subject's bodily changes, capable of informing dosage and duration, low cost, eliminates self-reporting data and provides a reliable biomarker.

The foregoing is illustrative of the present invention and is not to be construed as limiting thereof. The invention is defined by the following claims, with equivalents of the claims to be included therein.

1. A method of determining opioid responsiveness in a subject, the method comprising determining a metabolomic profile present in a sample from the subject comprising analysis of specific agents and/or biochemical pathways, wherein the biochemical pathways are selected from the group consisting of biochemical pathways involving tyrosine, tryptophan, linoleic acid, phenylalanine, sphingolipid, glycerophospholipid, D-glutamine, D-glutamate, dopamine and L-DOPA, and combinations thereof.

2. A method of overcoming opioid desensitization in a subject in need thereof, the method comprising determining a metabolomic profile present in a sample from the subject comprising analysis of specific agents and/or biochemical pathways, wherein the biochemical pathways are selected from the group consisting of biochemical pathways involving tyrosine, tryptophan, linoleic acid, phenylalanine, sphingolipid, glycerophospholipid, D-glutamine, D-glutamate, dopamine and L-DOPA, and combinations thereof.

3. The method of claim 1, wherein the biochemical pathway is a synthesis and/or metabolic pathway.

4. The method of claim 1, wherein the biochemical pathway is a dopamine-related synthesis and/or metabolic pathway.

5. The method of claim 1, wherein the biochemical pathway is a tyrosine-related synthesis and/or metabolic pathway.

6. The method of claim 1, wherein the subject is afflicted with or suspected of having acute pain, chronic pain, opioid-resistant neuropathic pain, and/or spinal cord injury related-pain.

7. The method of claim 1, wherein the subject is opioid responsive.

8. The method of claim 1, wherein the sample is a tissue sample, blood, serum, plasma, urine, sweat, saliva, tears, spinal fluid, stool, skin, hair and/or nails.

9. The method of claim 1, wherein opioid responsiveness includes likelihood of opioid addiction or dependence, opioid tolerance and/or efficacy.

10. A method of providing enhanced analgesia in a subject, the method comprising determining the metabolic profile present in a sample from the subject and administering to the subject an effective amount of an opioid when the subject is determined to be opioid responsive or suspected to be opioid responsive, or administering a non-opioid treatment when the subject is determined to be opioid non-responsive or suspected to be opioid non-responsive.

11. The method of claim 10, wherein the subject is opioid responsive.

12. The method of claim 11, wherein the subject is administered morphine.

13. The method of claim 11 further comprising administering to the subject an effective amount of a dopamine type 3 receptor (D3R) agonist.

14. The method of claim 13, wherein the subject is afflicted with or suspected of having acute pain, chronic pain, opioid-resistant neuropathic pain, or spinal cord injury related-pain.

15. A method of classifying a subject as opioid responsive or opioid non-responsive, the method comprising determining a metabolomic profile present in a sample from the subject comprising analysis of specific agents and/or biochemical pathways, wherein the biochemical pathways are selected from the group consisting of biochemical pathways involving tyrosine, tryptophan, linoleic acid, phenylalanine, sphingolipid, glycerophospholipid, D-glutamine, D-glutamate, dopamine and L-DOPA, and combinations thereof.

16. The method of claim 15, wherein the biochemical pathway is a synthesis and/or metabolic pathway.

17. The method of claim 15, wherein the biochemical pathway is a dopamine-related synthesis and/or metabolic pathway.

18. The method of claim 15, wherein the biochemical pathway is a tyrosine-related synthesis and/or metabolic pathway.

19. The method of claim 15, wherein the subject is afflicted with or suspected of having acute pain, chronic pain, opioid-resistant neuropathic pain, or spinal cord injury related-pain.

20. The method of claim 19 further comprising administering to the subject an effective amount of an opioid if the subject is determined to be opioid responsive or suspected to be opioid responsive, or administering a non-opioid treatment when the subject is determined to be opioid non-responsive or suspected to be opioid non-responsive.

21-24. (canceled)
